(19) United States
(12) Patent Application Publication
(10) Pub. No.: US 2003/0095936 A1
(43) Pub. Date: May 22, 2003

(54) Lip gloss composition

(76) Inventor: Orrea Light, New York, NY (US)

Correspondence Address: KATTEN MUCHIN ZAVIS ROSENMAN, 575 MADISON AVENUE, NEW YORK, NY, 10022-2585, US

(21) Appl. No.: 10/299,333

(22) Filed: Nov. 19, 2002

Related U.S. Application Data
(60) Provisional application No. 60/331,679, filed on Nov. 19, 2001.

(51) Int. Cl. A61K 7/025; A61K 7/06

(52) U.S. Cl. 424/64; 424/74

(57) ABSTRACT

A lip gloss composition that overcomes the sticky, oily and/or waxy feel of conventional lip gloss products, which applies softly, does not drag on application, and leaves the wearers' lips feeling soft, supple, comfortable, and protected against cracking and dryness.

### CROSS-REFERENCE TO RELATED APPLICATION

This application claims priority under 35 U.S.C. §119(e) from U.S. Provisional Patent application No. 60/331,679 filed on Nov. 19, 2001, and which is incorporated herein by reference.

### FIELD OF THE INVENTION

This invention relates to a lip gloss composition, and more particularly to a lip gloss composition that provides a beneficial moisturization effect, is easily applied on the lips, spreads smoothly and imparts increased cushion. This invention also applies to care and or treatment products for the lips and to make-up products for the lips which have care and/or treating properties.

### BACKGROUND OF INVENTION

Lip cosmetics were used by Syrians, Persians, Greeks and Romans for aesthetic, medical or ritualistic purposes.

Lip glosses, in general, are made of an oily vehicle comprising fat or oil stiffened to a desired consistency with waxes of various types, which serve to raise the melting point and improve the physical stability. Modem lip glosses comprise a base of oil and wax. Lip gloss bases may be categorized by chemical class (organic, silicone), source (natural, synthetic), or function (moisturizing, contouring).

Lip glosses generally comprise a solid fatty base in admixture with cosmetically acceptable waxes, oils, solids and semisolids. Waxy and oily materials are included in lip glosses to give the lips a moist and alluring look. Several of the most important materials used in lip gloss compositions are cosmetic waxes such as beeswax, ozokerite and cosmetic oils such as castor oil and lanolin. Beeswax adds binding and molding properties to lip gloss; Ozokerite gives lip gloss toughness; castor oil is a solvent for the dyes and functions as an emollient and lanolin aids in maintaining homogeneity during manufacturing as well as serving as an emollient. Emollients provide a supple and pleasant feeling to the lips of the wearer. Dyes, preservative and fragrance are typically also present.

Among the other numerous materials useful for incorporation into a lip gloss composition, fatty alcohols and fatty acid esters have been found to be useful in cosmetic products because of the ability of such compounds to maintain a porous fatty film on the lips.

Lips typically have a rough surface comprising tiny ridges and cracks. Such cracks detract from the smooth elegant appearance desired by most wearers. It is desirable to provide a lip gloss composition that fills these the tiny crevices on the surface of the lip and gives the wearer's lips a smoother more even surface. In addition, the small cracks, ridges and fissures typical are the initiation point of lip cracking and damage during dry and cold conditions.

It is also desirable to limit the greasy appearance in a make-up product for the lips. A sticky feel and a lack of slipperiness are drawbacks that are annoying and off-putting to the wearer. It is difficult at the present time to obtain colored lip products with treatment properties. The challenge is to allow the lips to be made up and protected aesthetically while at the same time treating them.

Thus, in addition to the cosmetic benefits of filling these tiny ridges and cracks to provide a smoother more elegant appearing look and feel, there exists the need to provide a lip gloss ridge and crack filling composition which provides a method of forestalling the presence of these ridges and cracks from acting as the nucleus of cracks and breakages of the skin during cold or dry weather.

### SUMMARY OF INVENTION

The present invention is a lip gloss formulation which is easily applied on the lips, spreads smoothly and imparts increased cushion providing a unique feel to the wearers lips while at the same time providing added protection against adverse environmental conditions. This composition can avoid drying-out, the formation of cracks and chapping, and can be useful for the treatment of cracks and chapping. It also makes the lips supple and soft. The gloss formula of this invention utilizes a novel wax base that imparts more moisturization to the lip, thereby providing a healing effect on chapped lips. The high cushion gels used in this gloss formula also provide a “cushion effect”.

This lip gloss imparts these properties by utilizing a gel/cream base incorporating a mixture of canola oil, Zea mays (corn) starch and silica and two botanicals as a synergistic complex to aide in moisturization. An important part of the composition is the use of a base system comprising the mixture of canola oil, Zea mays (corn) starch and silica to provide “extra cushion” and soft rich feel to the lip gloss.

### BRIEF DESCRIPTION OF THE DRAWINGS

FIG. 1 is a table of the ingredients of compositions within the scope of the present invention.

### DETAILED DESCRIPTION OF THE INVENTION

Lips typically have a rough surface comprising tiny ridges and cracks. It is desirable to provide a lip gloss composition that fills these the tiny crevices on the surface of the lip and gives the wearer's lips a smoother more even surface. A lip gloss base having high cushion gels incorporated into the product allows the product to fill in the rough areas. The high cushion gels in the disclosed composition, in combination with the other ingredients, deposit a thicker film on the lips and provide a smoother, more harmonious surface to the wearer's lips.

The disclosed lip gloss composition provides a unique feel and ease in application. It imparts cushion, creaminess, moisturization and substantivity (thicker film on lip) to the wearer thereby providing increased comfort, treating and ease in application to the consumer. This lip gloss imparts these properties by utilizing a gel/cream base incorporating a mixture of canola oil, Zea mays (corn) starch and silica and by utilizing two botanicals as a synergistic complex to aide in moisturization. A significant part of the composition is the use of a base system comprising the mixture of canola oil, Zea mays (corn) starch and silica to provide “extra cushion” and soft rich feel to the lip gloss. The composition utilizes a gel/cream base with incorporates canola gel, a proprietary mixture of canola oil, Zea mays (corn) starch and silica sold by Natunola Health (Nepean, Ontario, Canada) under its trademark Vegelatum® Clear.

Canola Oil is a vegetable oil from the canola seed that is very stable, high in oleic acid, rich in Vitamin E that is an excellent emollient and moisturizer and helps reduce skin irritation. Vegelatum® Clear is a botanical emollient produced from a non-transgenically modified canola oil.

Canola gel is superior to petroleum based emollients, such as petrolatum, as it has exceptional thermostability, a gel-like structure and is also less greasy. In combination with the other ingredients of the gel/cream base, it softens and smoothes the skin, forming a light film which prevents evaporation of moisture from the skin and protects the skin from irritation.

In appearance, the canola gel utilized is a clear gel, with a very light odor. It is soluble in all vegetable oils, glycerol triisostearate, isostearyl isosterate, oleic acid, isostearic acid, coco-caprylate/caprate and mixed glycerides and insoluble in water, absolute ethanol and 1,2-propanadiol, dimethicone and cyclomethicone. The base also incorporates beeswax, microcrystalline wax and ozokerite.

The composition of the present invention can also includes one or more oils or oil-like emollients. Any cosmetically or pharmaceutically acceptable oil may be used in the wax base, the selection only being limited by the necessity for successfully wetting out pigments, a technique well known in the art. Examples of suitable oils or oil-like emollients can be found in the International Cosmetic Ingredient Handbook, CTFA, 1996, the contents of which are incorporated herein by reference.

Useful materials include, but are not limited to, castor oil, coconut oil, corn oil, jojoba oil, cottonseed oil, soybean oil, walnut oil, wheat germ oil, sunflower seed oil, palm kernel oil, calendula oil, C8-18 triglycerides, lanolin and lanolin derivatives, illipe butter, shea butter; esters, such as isodecyl neopentanoate, tridecyl octanoate, diisostearyl malate, cetyl palmitate, cetyl octanoate, cetyl stearate, cetyl myristate, isopropyl palmitate, isopropyl myristate, dipentaerythrityl hexahydroxy stearate/stearate/rosinate, polyglyceryl-2-isostearate, neopentyl glycol distearate, isodecyl oleate, decyl isostearate, diisopropyl sebacate, PEG-4 diheptanoate, dioctyl malate, and isohexyl neopentanoate; fatty alcohols, such as lanolin alcohol or oleyl alcohol; and silicone oils, such as cyclomethicone, dimethicone, cetyl dimethicone, lauryl trimethicone, and dimethiconol.

There are a number of other ingredients approved for use in the cosmetic art that may be used in compositions of the present invention. Such ingredients are those approved for use in cosmetics and can be found listed in reference books such as the CTFA Cosmetic Ingredient Handbook, Second Edition, The Cosmetic, Toiletries, and Fragrance Association, Inc. 1988, 1992. Said materials may be used provided their inclusion does not significantly disrupt the composition once it has been applied to the skin wherein a film has been formed. Said ingredients include waxes, fragrances, flavor oils, skin care ingredients such as sunscreen, emulsifiers and the like. Hypoallergenic compositions can be made into the present invention where said compositions do not contain fragrances, flavor oils, lanolin, sunscreens, particularly PABA, or other sensitizers and irritants.

Waxes used in the present invention are used at levels that do not interfere with film formation process. Generally waxes are not used in the present invention higher than about 10% of the composition, preferably not higher than about 8% of the composition. Waxes are defined as lower-melting organic mixtures or compounds of high molecular weight, solid at room temperature and generally similar in composition to fats and oils except that they contain no glycerides. Some are hydrocarbons, others are esters of fatty acids and alcohols. Waxes useful in the present invention are selected from the group consisting of animal waxes, vegetable waxes, mineral waxes, various fractions of natural waxes, synthetic waxes, petroleum waxes, ethylenic polymers, hydrocarbon types such as Fischer-Tropsch waxes, silicone waxes, and mixtures thereof. Waxes preferred for use in the present invention are selected from the group consisting of microcrystalline wax, Ozokerite and beeswax.

Flavor oils such as peppermint oil, orange oil, citrus oil, or wintergreen oil can be used along with an alcohol or glycerine. Flavor oils are usually mixed in a solvent such as ethanol to dilute the flavor. The flavor oils useful herein can be derived from natural sources or be synthetically prepared. Generally, flavor oils are mixtures of ketones, alcohols, fatty acids, esters and terpenes. The term “flavor oil” is generally recognized in the art to be a liquid which is derived from botanical sources, i.e. leaves, bark, or skin of fruits or vegetables, and which are usually insoluble in water. The level of flavor oil used can range from 0% to about 0.5%, preferably from 0% to about 0.3% of the lip composition.

Emulsifiers may be used as coupling agents that have an affinity for the hydrophilic and hydrophobic phases of lip compositions of this invention. Such emulsifiers include those routinely used in cosmetics and are found in the CTFA.

Skin care active ingredients in both water-soluble and water insoluble forms can be added to the lip gloss composition. Said ingredients may include fat-soluble vitamins, sunscreens and pharmaceutically active ingredients. Skin care active ingredients include glycerine, zinc oxide; chamomile oil; ginko biloba extract; pyroglutamic acid, salts or esters; sodium hyaluronate; 2-hydroxyoctanoic acid; sulfur; salicylic acid; carboxymethyl cysteine, water, propylene glycol and mixtures thereof.

The most important parts of the composition and those which provide the superior qualities are the gels in the base, in conjunction with the synergistic effect of Shea butter, and Prunus oil. Prunus oil aids in hydration and dryness prevention. Prunus oil is further recognized scientifically as a free-radical scavenger.

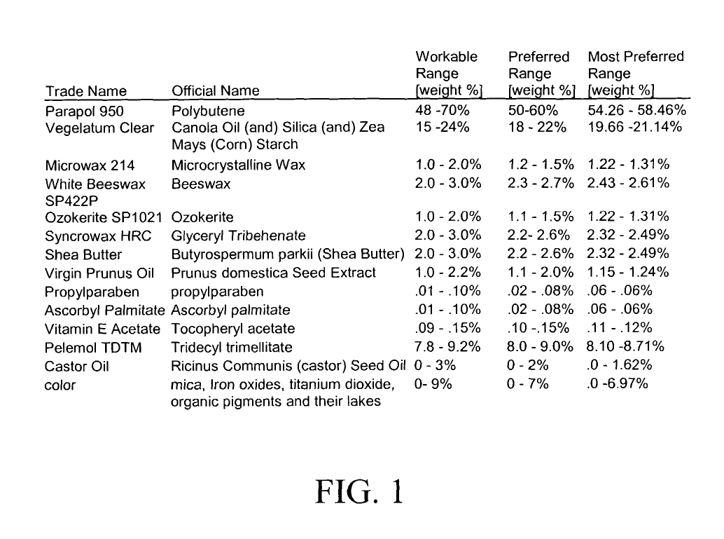

It is the use of these ingredients, in the specified proportions and alone or in combination with the other listed ingredients, all as set forth in the table of FIG. 1, that yields the superior results obtained. The table of FIG. 1 describes the ingredients most useful in compositions of this invention along with preferred embodiments of the inventive composition.

In a most preferred embodiment, the disclosed lip gloss compositions imparting increased cushion, creaminess, moisturization and substantivity comprise polybutene; mixture of canola oil, silica and Zea mays (corn) starch; microcrystalline wax; beeswax; ozokerite; glyceryl tribehenate; Butyrospermum parkii (Shea Butter); Prunus domestica seed extract; propylparaben; ascorbyl palmitate; tocopheryl acetate; tridecyl trimellitate; and Ricinus communis (castor) seed oil.

I claim:

1. A lip gloss composition imparting increased cushion, creaminess, moisturization and substantivity comprising vegelatum clear and virgin prunus oil.

2. The lip gloss composition of claim 1 comprising vegelatum clear in an amount of from about 15 to about 24 weight % and virgin prunus oil in an amount of from about 1 to about 2.2 weight %.

3. The lip gloss composition of claim 2 comprising vegelatum clear in an amount of from about 18 to about 22 weight % and virgin prunus oil in an amount of from about 1.1 to about 2 weight %.

4. The lip gloss composition of claim 2 further comprising tridecyl trimellitate in an amount of from about 7.8 to about 9.2 weight %.

5. The lip gloss composition of claim 2 further comprising tridecyl trimellitate in an amount of from about 8 to about 9 weight %.

6. A lip gloss composition imparting increased cushion, creaminess, moisturization and substantivity comprising polybutene; mixture of canola oil, silica and Zea mays (corn) starch; microcrystalline wax; beeswax; ozokerite; glyceryl tribehenate; Butyrospermum parkii (Shea Butter); Prunus domestica seed extract; propylparaben; ascorbyl palmitate; tocopheryl acetate; tridecyl trimellitate; and Ricinus communis (castor) seed oil.

7. The lip gloss composition of claim 6 additionally comprising fragrance and/or flavor components.

8. The lip gloss composition of claim 6 comprising:

| | Minimum | Maximum |
|---|---|---|
| Official Name | [weight %] | [weight %] |
| Polybutene | 48 | 70 |
| Canola Oil/Silica/Zea Mays (Corn) Starch | 15 | 24 |
| Microcrystalline Wax | 1.0 | 2.0 |
| Beeswax | 2.0 | 3.0 |
| Ozokerite | 1.0 | 2.0 |
| Glyceryl tribehenate | 2.0 | 3.0 |
| Butyrospermum parkii (Shea Butter) | 2.0 | 3.0 |
| Prunus domestica Seed Extract | 1.0 | 2.2 |
| propylparaben | 0.01 | 0.10 |
| Ascorbyl palmitate | 0.01 | 0.10 |
| Tocopheryl acetate | 0.09 | 0.15 |
| Tridecyl trimellitate | 7.8 | 9.2 |
| Ricinus communis (castor) Seed Oil | 0 | 3 |
| mica, iron oxides, titanium dioxide, organic | 0 | 9 |
| pigments and their lakes | | |

9. The lip gloss composition of claim 6 comprising:

| | Minimum | Maximum |
|---|---|---|
| Official Name | [weight %] | [weight %] |
| Polybutene | 50 | 60 |
| Canola Oil/Silica/Zea Mays (Corn) Starch | 18 | 22 |
| Microcrystalline Wax | 1.2 | 1.5 |
| Beeswax | 2.3 | 2.7 |
| Ozokerite | 1.1 | 1.5 |
| Glyceryl tribehenate | 2.2 | 2.6 |
| Butyrospermum parkii (Shea Butter) | 2.2 | 2.6 |
| Prunus domestica Seed Extract | 1.1 | 2.0 |
| propylparaben | 0.02 | 0.08 |
| Ascorbyl palmitate | 0.02 | 0.08 |
| Tocopheryl acetate | 0.10 | 0.15 |
| Tridecyl trimellitate | 8.0 | 9.0 |
| Ricinus communis (castor) Seed Oil | 0 | 2 |
| mica, iron oxides, titanium dioxide, organic | 0 | 7 |
| pigments and their lakes | | |

10. The lip gloss composition of claim 6 comprising:

| | Minimum | Maximum |
|---|---|---|
| Official Name | [weight %] | [weight %] |
| Polybutene | 54.26 | 58.46 |
| Canola Oil/Silica/Zea Mays (Corn) Starch | 19.66 | 21.14 |
| Microcrystalline Wax | 1.22 | 1.31 |
| Beeswax | 2.43 | 2.61 |
| Ozokerite | 1.22 | 1.31 |
| Glyceryl tribehenate | 2.32 | 2.49 |
| Butyrospermum parkii (Shea Butter) | 2.32 | 2.49 |
| Prunus domestica Seed Extract | 1.15 | 1.24 |
| propylparaben | 0.06 | 0.06 |
| Ascorbyl palmitate | 0.06 | 0.06 |
| Tocopheryl acetate | 0.11 | 0.12 |
| Tridecyl trimellitate | 8.10 | 8.71 |
| Ricinus communis (castor) Seed Oil | 0 | 1.62 |
| mica, iron oxides, titanium dioxide, organic | 0 | 6.97 |
| pigments and their lakes | | |
